(19) United States
(12) Patent Application Publication
(10) Pub. No.: US 2015/0025524 A1
(43) Pub. Date: Jan. 22, 2015

(54) RENAL DENERVATION MONITORING AND FEEDBACK APPARATUS, SYSTEM AND METHOD

(71) Applicant: ST. JUDE MEDICAL, CARDIOLOGY DIVISION, INC., St. Paul, MN (US)

(72) Inventor: Yelena Nabutovsky, Sunnyvale, CA (US)

(21) Appl. No.: 14/320,990

(22) Filed: Jul. 1, 2014

Related U.S. Application Data
(60) Provisional application No. 61/847,652, filed on Jul. 18, 2013.

(51) Int. Cl. A61B 18/14

(52) U.S. Cl. CPC A61B 18/1492; A61B 2018/00434
USPC 606/34

(57) ABSTRACT

Methods, systems, and apparatuses for measuring the efficacy of a renal denervation procedure are described. In one embodiment, a method for measuring the efficacy of a renal denervation procedure includes utilizing an ablation catheter including one or more sensors designed and configured for measuring and reporting the plasma norepinephrine level of a subject. The plasma norepinephrine levels of the subject may be measured intermittently after each individual ablation, or may be continuously monitored throughout an entire ablation procedure to provide real time plasma norepinephrine level data to the operator to assist in determining the efficacy of the procedure.

### CROSS-REFERENCE TO RELATED APPLICATIONS

This application claims priority to provisional application Ser. No. 61/847,652, filed Jul. 18, 2013, the entire specification of which is incorporated herein.

### BACKGROUND OF THE DISCLOSURE

a. Field of the Disclosure

The present disclosure relates generally to methods, systems, and apparatuses for monitoring and assessing renal denervation procedures in real time. More particularly, the present disclosure relates to monitoring and assessing methods, systems, and apparatuses that utilize a specific sensor or sensors to measure, monitor, and/or record/report plasma norepinephrine levels before, during, and/or after renal denervation procedures to assess the progress and success of the individual ablations and the procedure.

b. Background Art

It is known that various renal ablation procedures for the ablation of perivascular renal nerves have been used for the treatment of hypertension, and specifically for drug-resistant hypertension. Generally, one or more radiofrequency electrodes are introduced into the body and fed into the renal artery and used to ablate the efferent and afferent nerves that generally run the length of the artery. In some cases, a single ablation procedure may include six to ten or more ablation areas along and around the wall of the artery. Typically, the operator performing the procedure will ablate one discrete area of the artery and then move the ablation electrode a desired distance lengthwise about the length of the artery and also rotate the handle of the catheter to move the ablation electrode circumferentially around the artery. In some cases, the operator may move the ablation electrode circumferentially about 45 degrees around the artery wall between ablations. By varying the ablation treatment sites lengthwise down and circumferentially around the artery wall, any potential overall damage to the artery wall can be minimized or eliminated while the overall ablation of the efferent and afferent nerves can still be substantially complete and effective.

During the ablation procedure, the operator, typically a doctor, performing the procedure generally attempts to monitor and track all of the areas of the artery wall that have previously been ablated to avoid over-treatment of any one site. This monitoring and tracking should be done both along the length of the artery as well as around the circumference of the artery wall to ensure proper ablation of the arterial nerves and the best procedural results. Feedback to the operator is generally provided regarding the temperature at the ablation site, which can be indicative of the effectiveness of the ablation itself, and whether the nerve has been ablated.

Based on the foregoing, it would be advantageous to provide improved methods, systems, and apparatuses for providing real time operational feedback as to the success of the ablation and its overall effectiveness on reducing blood pressure to the operator of the renal denervation system to allow for more precise and thorough ablation of a renal artery. It would also be advantageous if the means for providing the real time feedback could be easily incorporated into or onto conventional renal denervation catheters or renal denervation equipment and could be used periodically or continuously throughout the renal denervation procedure to provide the real time data to the operator regarding the effectiveness of the ablations being performed such that the procedure can be modified accordingly.

### BRIEF SUMMARY OF THE DISCLOSURE

In one aspect, the present disclosure is directed to a method of measuring the efficacy of a renal denervation procedure on a subject. The method comprises measuring one or more plasma norepinephrine levels of the subject during the renal denervation procedure.

In another aspect, the present disclosure is directed to a method of monitoring the progress of a renal denervation procedure on a subject. The method comprises first measuring a baseline plasma norepinephrine level and then performing an ablation and measuring a post-ablation plasma norepinephrine level.

Another aspect of the present disclosure is directed to a method of monitoring the progress of a renal denervation procedure on a subject. The method comprises first measuring a baseline plasma norepinephrine level of the subject and then performing one or more ablations on the subject while continuously monitoring the plasma norepinephrine level of the subject.

Another aspect of the present disclosure is directed to an ablation catheter comprising an elongated catheter body having a distal end and a proximal end, and one or more electrodes located near the distal end of the elongated catheter body. The ablation catheter further comprises at least one sensor for measuring plasma norepinephrine levels.

### BRIEF DESCRIPTION OF THE DRAWINGS

FIG. 1 is a diagrammatic view of a renal denervation system for presenting information relating to lesion formation in a renal artery in accordance with embodiments of the present disclosure.

FIG. 2 is a diagram of an ablation catheter including an electrode basket and plasma norepinephrine sensors.

Corresponding reference characters indicate corresponding parts throughout the drawings.

### DETAILED DESCRIPTION OF THE DISCLOSURE

During hypertension, there are increased levels of the catecholamine neurotransmitter norepinephrine in the plasma. Norepinephrine mediates chemical communication in the sympathetic nervous system and, like other neurotransmitters, is released at synaptic nerve endings to transmit the signal from a nerve cell to other cells. Norepinephrine is an indicator of a stress response, and high levels of norepinephrine have been associated with increased mortality. One of the desired effects of renal denervation is the disruption of the sympathetic nervous system feedback, which results in decreased hypertension and hence, a decrease of the plasma levels of norepinephrine.

The present disclosure is generally directed to monitoring the plasma levels of the neurotransmitter norepinephrine before, during, and/or after a renal denervation procedure on a subject. More specifically, the present disclosure is directed to apparatuses, methods and systems for assessing, monitoring, measuring and/or recording/reporting plasma norepinephrine levels before, during, and/or after a renal denervation procedure that may include one or more ablations on a subject. Each ablation performed on the subject may be of the same or different duration, and may be of the same or different power level. Apparatuses used in accordance with the present disclosure may include one or more electrodes, which may be located on an electrode basket as described herein, that may have attached thereto one or more sensors capable of measuring plasma norepinephrine levels. Alternatively, the sensor or sensors may be otherwise located on a separate catheter or the like. In addition to measuring the plasma norepinephrine levels, the one or more sensors may be configured to report this data to an operator of the renal denervation system.

In accordance with other method embodiments of the present disclosure, the norepinephrine levels of plasma may be monitored intermittently or periodically before, during, and/or after renal denervation ablations, or may be continuously monitored before, during, and/or after renal denervation ablations. In many embodiments described herein, a baseline plasma norepinephrine level is determined prior to any ablations taking place, followed by one or more ablations and monitoring of the plasma norepinephrine levels to assess the effect and completeness of the renal denervation procedure.

The various approaches described herein may allow an ablation catheter system to measure the efficacy and/or success of renal efferent and afferent nerve ablation in real time during a renal denervation procedure to provide immediate success feedback to an operator or doctor throughout an ablation procedure. With this real time information, the system operator can minimize both the number of ablations performed and the energy used. This can reduce or eliminate any potential damage to the renal artery and improve patient outcomes. Additionally, the real time information may allow for improved overall procedure management and efficiency. These and other benefits of the disclosure are set forth in detail herein.

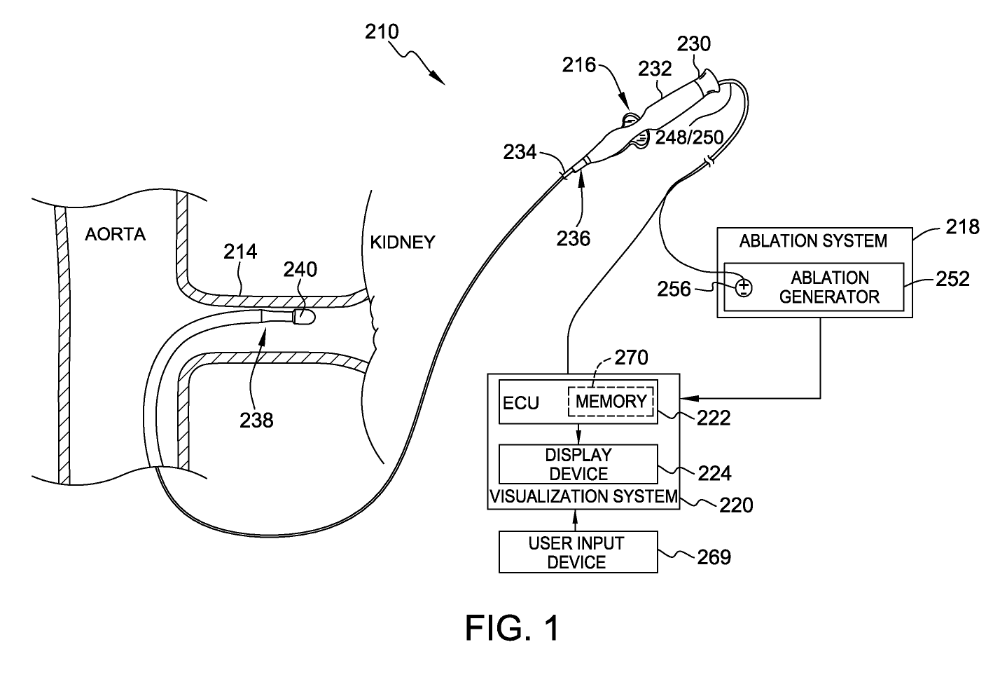

Referring now to the Figures, FIG. 1 illustrates one exemplary embodiment of an ablation system 210 for performing one or more diagnostic and/or therapeutic functions that include components for presenting information representative of lesion formations in renal artery 214 during an ablation procedure performed thereon. It should be understood, however, that system 210 has equal applicability to ablation procedures on other tissues as well, including cardiac tissues.

Among other components, system 210 includes a medical device (such as, for example, catheter 216), ablation system 218, and system 220 for the visualization, navigation, and/or mapping of internal body structures. System 220 may include, for example and without limitation, an electronic control unit (ECU) 222, display device 224, user input device 269, and memory 270. Alternatively, ECU 222 and/or display 224 may be separate and distinct from, but electrically connected to and configured for communication with, system 220.

With continued reference to FIG. 1, catheter 216 is provided for examination, diagnosis, and/or treatment of internal body tissues, such as renal artery 214. In an exemplary embodiment, catheter 216 comprises a radio frequency (RF) ablation catheter. It should be understood, however, that catheter 216 is not limited to an RF ablation catheter. Rather, in other embodiments, catheter 216 may comprise an irrigated catheter and/or other types of ablation catheters (e.g., cryoablation, ultrasound, etc.).

In an exemplary embodiment, catheter 216 is electrically connected to ablation system 218 to allow for the delivery of RF energy. Catheter 216 may include a cable connector or interface 230, handle 232, shaft 234 having a proximal end 236 and distal end 238 (as used herein, “proximal” refers to a direction toward the end of catheter 216 near the operator, and “distal” refers to a direction away from the operator and (generally) inside the body of a subject or patient), and one or more electrodes 240 mounted in or on shaft 234 of catheter 216. In an exemplary embodiment, electrode 240 is disposed at or near distal end 238 of shaft 234, with electrode 240 comprising an ablation electrode disposed at the extreme distal end 238 of shaft 234 for contact with renal artery 214. Catheter 216 may further include other conventional components such as, for example and without limitation, sensors, additional electrodes (e.g., ring electrodes) and corresponding conductors or leads, or additional ablation elements, e.g., a high intensity focused ultrasound ablation element and the like.

Connector 230 provides mechanical and electrical connection(s) for cables 248 and 250 extending from ablation system 218, and visualization, navigation, and/or mapping system 220. Connector 230 is conventional in the art and is disposed at the proximal end of catheter 216.

Handle 232 provides a location for the operator to hold catheter 216 and may further provide means for steering or guiding shaft 234 within renal artery 214. For example, handle 232 may include means to change the length of a guidewire extending through catheter 216 to distal end 238 of shaft 234 to steer shaft 234. Handle 232 is also conventional in the art and it will be understood that the construction of handle 232 may vary. In another exemplary embodiment, catheter 216 may be robotically driven or controlled. Accordingly, rather than an operator manipulating a handle to steer or guide catheter 216, and shaft 234 thereof, in particular, a robot is used to manipulate catheter 216.

Shaft 234 is generally an elongated, tubular, flexible member configured for movement within renal artery 214. Shaft 234 supports, for example and without limitation, electrode 240, associated conductors, and possibly additional electronics used for signal processing or conditioning. Shaft 234 may also permit transport, delivery and/or removal of fluids (including irrigation fluids, cryogenic ablation fluids, and bodily fluids), medicines, and/or surgical tools or instruments. Shaft 234 may be made from conventional materials such as polyurethane, and defines one or more lumens configured to house and/or transport at least electrical conductors, fluids, or surgical tools. Shaft 234 may be introduced into renal artery 214 through a conventional introducer. Shaft 234 may then be steered or guided through renal artery 214 to a desired location with guidewires or other means known in the art.

With further reference to FIG. 1, ablation system 218 is comprised of, for example, ablation generator 252. Ablation generator 252 generates, delivers, and controls RF energy output by ablation catheter 216 and electrode 240 thereof, in particular. In an exemplary embodiment, generator 252 includes RF ablation signal source 256 configured to generate an ablation signal that is output across a pair of source connectors: a positive polarity connector SOURCE (+), which may be electrically connected to tip electrode 240 of catheter 216; and a negative polarity connector SOURCE (−). It should be understood that the term connectors as used herein does not imply a particular type of physical interface mechanism, but is rather broadly contemplated to represent one or more electrical nodes. Source 256 is configured to generate a signal at a predetermined frequency in accordance with one or more user specified parameters (e.g., power, time, etc.) and under the control of various feedback sensing and control circuitry as is known in the art. Source 256 may generate a signal, for example, with a frequency of about 450 kHz or greater. Generator 252 may also monitor various parameters associated with the ablation procedure including, for example, impedance, the temperature at the distal tip of the catheter, applied ablation energy, and the position of the catheter, and provide feedback to the clinician or another component within system 210 regarding these parameters.

In accordance with the present disclosure, the ablation system described above may additionally include one or more sensitive and selective biocompatible sensors designed and configured to detect, measure, assess, monitor and/or report the plasma norepinephrine levels of a subject before, during, and/or after one or more ablations, and particularly after one or more ablations of a renal artery. Suitable sensors include, for example, electrochemical sensors and the like. Each sensor or sensors used in accordance with the present disclosure for plasma norepinephrine level determination may include one, two, three or more individual electrodes working separately or together and configured to measure and/or record/report plasma norepinephrine levels using, for example, square wave voltammetry and/or cyclic voltammetry. Other types of voltammetry or other electrical measurement methods are also within the scope of the present disclosure.

In one specific embodiment, each sensor or sensors included in the ablation system may include a combination of three separate and distinct electrodes including a working electrode (as described more fully below), a counter electrode (such as a platinum wire counter electrode, for example), and a reference electrode (such as a mercury chloride or silver chloride reference electrode, for example). In some embodiments of the present disclosure, the sensor or sensors may be located on or near the electrode tip that is inserted into the renal artery during a renal denervation procedure, and particularly when the electrode tip is in the form of a conventional electrode basket, as is known in the art. The sensor or sensors may be located at any position or positions on the electrode basket (or any other form of electrode tip) suitable for holding one or more sensors in place during an ablation procedure. The one or more sensors may be located adjacent to, or away from, the one or more electrodes used for ablation. Other types of sensors (i.e., pressure sensors, temperature sensors, etc.) may also be present on the electrode basket (or any other form of electrode tip) in combination with the one or more plasma norepinephrine sensor or sensors and the one or more electrodes.

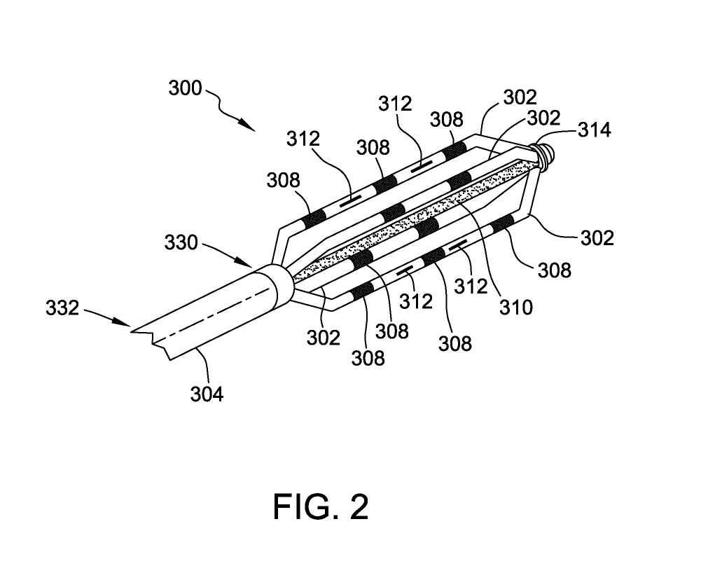

Referring now to FIG. 2, there is shown an exemplary electrode basket 300 that is suitable for including one or more plasma norepinephrine sensors as described herein. Electrode basket 300 includes a plurality of splines 302 connected to an elongated catheter body 304 having distal end 330 and proximal end 332. A longitudinal rod 310 is located in the center of the electrode basket 300 and is attached to junction 314. Longitudinal rod 310 can be used to expand/contract electrode basket 300 into different conformations before, during, and after an ablation procedure. Splines 302 of electrode basket 300 include ablation electrodes 308, as well as plasma norepinephrine sensors 312.

Alternatively, in other embodiments in accordance with the present disclosure, the plasma norepinephrine sensor or sensors may be otherwise located on, for example, an additional catheter that is inserted into the renal artery during a renal denervation procedure such that the sensor or sensors are not located on the electrode tip or basket. This dual catheter system may be desirable in some embodiments where, for example, the electrode basket includes numerous other sensors in addition to the electrodes. The electrode tip or basket, or other catheter, may include only a single sensor for measuring the plasma norepinephrine level, or may include two or more sensors for measuring the plasma norepinephrine levels wherein the readings from each individual sensor may optionally be averaged and reported to the operator as a single averaged value. Alternatively, the plasma norepinephrine sensor or sensors may be located in another area or areas of the ablation system. The exact location of the one or more plasma norepinephrine sensors is not critical, so long as the sensor or sensors can contact the blood of the subject, and hence the plasma that contains the norepinephrine to allow for a norepinephrine level reading.

The plasma norepinephrine sensor or sensors may be sized and configured such that they are wired to convey plasma norepinephrine levels to a readable and/or audible medium before, during, and/or after a renal denervation procedure. One suitable readable medium may be, for example, a display device such as a computer screen, monitor, or the like. Alternatively, the plasma norepinephrine sensor or sensors may be sized and configured to wirelessly convey plasma norepinephrine levels to a readable and/or audible medium before, during, and/or after a renal denervation procedure.

In one specific embodiment, the sensor or sensors for measuring and/or recording plasma norepinephrine levels may include a clenbuterol modified paraffin-impregnated graphite electrode as the working electrode. In one exemplary embodiment, the clenbuterol modified paraffin-impregnated graphite electrode is used in combination with a platinum wire counter electrode and a saturated calomel reference electrode. In some embodiments, the clenbuterol modified paraffin-impregnated graphite electrode may have a formal surface area of about 0.125 cm², although smaller and larger formal surface areas are within the scope of the present disclosure. The formal surface area may change based upon the specific placement of the sensor or sensors within the ablation system. Suitable clenbuterol modified paraffin-impregnated graphite electrodes for use as plasma norepinephrine sensors in the present disclosure are disclosed, for example, by Jin et al., Biosensors and Bioelectronics 24 (2008) 1031-1035, the disclosure of which is incorporated herein by reference.

In another specific embodiment, the sensor or sensors for measuring and/or recording plasma norepinephrine levels may include a nanogold-based electrochemical electrode as the working electrode. In one exemplary embodiment, the nanogold-based electrochemical electrode may be used in combination with a platinum wire counter electrode and a silver/silver chloride reference electrode. In some embodiments, the nanogold-based electrode may have a formal surface area of about 0.125 cm², although smaller and larger formal surface areas are within the scope of the present disclosure. In some particular embodiments, a suitable nanogold-based electrode may include a stable layer of gold nanoparticles deposited onto the surface of indium tin oxide. Suitable nanogold-based electrodes for use as plasma norepinephrine sensors in the present disclosure are disclosed, for example, by Goyal et al., Sensors and Actuators B 153 (2011) 232-238, the disclosure of which is incorporated herein by reference.

In one exemplary embodiment of the present disclosure, the norepinephrine sensor or sensors described above are utilized in a renal denervation procedure (or other ablation procedure, such as, for example, cardiac ablation) to intermittently measure and/or record/report plasma norepinephrine levels to the renal denervation operator such that the progress and success of the ablations can be monitored and assessed; that is, the sensors are used in an “on-again, off-again” process to provide plasma norepinephrine levels to the system operator on a periodic, discontinuous basis to determine the efficacy of the ablations and overall procedure. In one embodiment of this process, the operator may first determine a baseline plasma norepinephrine level of the subject; that is, the operator may determine the plasma norepinephrine level of the subject prior to any ablations being performed such that the operator has a baseline level plasma norepinephrine level from which to interpret future plasma norepinephrine level readings. Alternatively, the baseline plasma norepinephrine level may be determined in another manner prior to the renal denervation procedure and the plasma norepinephrine sensor or sensors solely used as described below. In other embodiments, a baseline plasma norepinephrine value may not be determined

Once the baseline plasma norepinephrine level of the subject has been determined, in some embodiments the operator may determine a target percent reduction in norepinephrine level that will be the endpoint of the renal denervation procedure; that is, a percent reduction target in plasma norepinephrine level may be determined as compared to the baseline level and ablations performed until the percent reduction target is generally achieved. In some embodiments, a target percent reduction in plasma norepinephrine level may be about 90%, or about 80%, or about 70%, or about 60% or about 50%, or about 40%, or about 30%, or about 20%, or even about 15% or even about 10% as compared to the baseline, pre-ablation plasma norepinephrine level. Alternatively, in other embodiments, the operator may choose an absolute value number as a target as compared to the baseline value and the ablations may proceed until the absolute target value is reached. The absolute target value may be, for example, a known plasma norepinephrine value for healthy, non-hypertensive, adults.

In these above-described embodiments, by receiving the plasma norepinephrine levels from the sensors after each successive ablation of the renal artery, the operator can judge the success and result of each individual ablation and determine whether additional ablations, or additional ablations with lower/higher energy and/or shorter/longer pulse width, should be performed to achieve the desired plasma norepinephrine values. Although the operator may generally obtain plasma norepinephrine levels after each successive ablation, it is within the scope of the present disclosure to determine plasma norepinephrine readings after two, three, four, or more ablations.

In an alternative embodiment of the present disclosure, after a baseline plasma norepinephrine level has been determined as described above, the plasma norepinephrine level of the subject may be measured continuously such that the operator has a continuous, uninterrupted stream of plasma norepinephrine level data throughout a renal denervation procedure; that is, instead of monitoring the plasma norepinephrine levels intermittently and periodically as described above, the one or more sensors are continually active such that plasma norepinephrine level data is continuously provided to the operator to determine the efficacy of the ablations and the overall procedure. In this embodiment, the energy of each subsequent ablation pulse, along with the pulse duration, can be increased or decreased depending upon the response of the plasma norepinephrine level to the latest ablation. Also, in this embodiment, the operator receives constant, continuous real time data for the plasma norepinephrine level of the subject, which is indicative of the procedural success.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above without departing from the scope of the disclosure, it is intended that all matter contained in the above description and shown in the accompanying drawings shall be interpreted as illustrative and not in a limiting sense.

What is claimed is:

1. A method of measuring the efficacy of a renal denervation procedure on a subject, the method comprising measuring one or more plasma norepinephrine levels of the subject during the renal denervation procedure.

2. The method of claim 1 wherein the renal denervation procedure includes one or more ablations and wherein a baseline plasma norepinephrine level is measured prior to any ablation occurring.

3. The method of claim 2 wherein the plasma norepinephrine level is measured after one or more ablations.

4. The method of claim 3 wherein the plasma norepinephrine level is communicated to an operator after each measurement.

5. The method of claim 1 wherein the plasma norepinephrine level is continuously monitored during the renal denervation procedure.

6. The method of claim 1 wherein the plasma norepinephrine level is periodically measured during the renal denervation procedure.

7. The method of claim 1 wherein the plasma norepinephrine level is measured using an electrochemical sensor.

8. The method of claim 7 wherein the electrochemical sensor is a nanogold-based sensor.

9. The method of claim 1 wherein the plasma norepinephrine level is measured using a clenbuterol-modified paraffin-impregnated graphite electrode.

10. The method of claim 9 wherein the clenbuterol-modified paraffin-impregnated graphite electrode is located on an ablation electrode.

11. A method of monitoring the progress of a renal denervation procedure on a subject, the method comprising:
measuring a baseline plasma norepinephrine level of the subject;
performing an ablation; and
measuring a post-ablation plasma norepinephrine level of the subject.

12. The method of claim 11 wherein the multiple ablations and multiple measurements are performed until a target norepinephrine level is measured.

13. The method of claim 11 wherein the plasma norepinephrine level is communicated to an operator after each measurement.

14. The method of claim 11 wherein the plasma norepinephrine level is measured using an electrochemical sensor.

15. The method of claim 14 wherein the electrochemical sensor is a nanogold-based sensor.

16. The method of claim 11 wherein the plasma norepinephrine level is measured using a clenbuterol-modified paraffin-impregnated graphite electrode.

17. The method of claim 16 wherein the clenbuterol-modified paraffin-impregnated graphite electrode is located on an ablation electrode.

18. A method of monitoring the progress of a renal denervation procedure on a subject, the method comprising:
measuring a baseline plasma norepinephrine level of the subject; and
performing one or more ablations on the subject while continuously monitoring the plasma norepinephrine level of the subject.

19. The method of claim 18 wherein the plasma norepinephrine level is measured using a nanogold-based sensor.

20. The method of claim 18 wherein the plasma norepinephrine level is measured using a clenbuterol-modified paraffin-impregnated graphite electrode.

21. An ablation catheter comprising an elongated catheter body having a distal end and a proximal end, and an electrode located near the distal end of the elongated catheter body, wherein the ablation catheter further comprises at least one sensor for measuring plasma norepinephrine levels.
